(19) United States
(12) Patent Application Publication
(10) Pub. No.: US 2026/0115426 A1
(43) Pub. Date: Apr. 30, 2026

(54) SUPPORT STRUCTURE FOR MEDICAL APPARATUS AND METHOD OF MANUFACTURING SAME

(71) Applicant: Canon U.S.A., Inc., Melville, NY (US)

(72) Inventor: Matthew Michael Kincaid, Medford, MA (US)

(21) Appl. No.: 19/427,368

(22) Filed: Dec. 19, 2025

Related U.S. Application Data
(62) Division of application No. 17/287,958, filed on Apr. 22, 2021, now Pat. No. 12,515,017.
(60) Provisional application No. 62/752,219, filed on Oct. 29, 2018.

(51) Int. Cl. A61M 25/01; A61M 25/00

(52) U.S. Cl. CPC A61M 25/0147; A61M 25/0108; A61M 2025/0037; A61M 2025/0059; A61M 2025/015

(57) ABSTRACT

Provided are an apparatus and method for use of same, the apparatus including a bendable body including a hollow extending a length thereof, with the hollow having a wall formed about at least a part thereof; and at least one support wire extending in the hollow. A distal end of the at least one support wire is anchored to the wall, with proximal portions of the at least one support wire being slideably situated in the hollow. At the proximal end of the bendable body, a proximal end of the at least one support wire is attached to at least one of a spring and a force controller. The at least one support wire provides a restoring force to prevent kinking of the bendable body.

### PRIORITY

This application claims priority to U.S Provisional Patent Application No. 62/752,219, filed on October 29, 2018, and PCT/US2019/057705, filed October 23, 2019, and is a Divisional Application of U.S. Patent Application No. 17/287,958, filed April 22, 2021, the entire disclosure of each of which is incorporated herein by reference.

### FIELD

The present disclosure relates generally to support structures for a medical apparatus and methods for manufacturing and incorporating the support structures. More particularly, the subject disclosure is directed to an articulated medical device having a hollow central cavity, wherein the device is capable of maneuvering within a patient without kinking. The support structure is instrumental in providing resiliency to the medical apparatus such that the medical apparatus does not kink or buckle when articulated. Exemplary uses for the medical apparatus may include endoscopes, cameras, and catheters.

### BACKGROUND

Bendable medical instruments such as endoscopic surgical instruments and catheters are well known and continue to gain acceptance in the medical field. The bendable medical instruments generally include flexible tubes commonly referred to as a sleeves or sheaths. One or more tool channels extend along (typically inside) the sheath to allow access to a target located at a distal end of the sheath.

The medical instruments are intended to provide flexible access within a patient, with at least one curve or more leading to the intended target, while retaining torsional and longitudinal rigidity so that a physician can control the tool being manipulated at the distal end of the medical instrument.

Recently, to enhance maneuverability of the distal end of the instrument, robotized instruments (a/k/a robots) that control distal portions have emerged. In general, these robots are elongated instruments that are meant to be steerable through tortuous pathways and around objects to arrive at some desired location. The medical devices detailed herein are usable for insertion down a patient’s airway, through the trachea and into the lungs. However, the subject innovation can obviously be employed in various other circumstances and anatomical fissures.

Once there, the purpose of the robot, or steerable catheter, is to reach an area of interest and to provide a working channel for tools such as a biopsy forceps, which can be used to sample the local tissue. To reach the area of interest, the medical device must be flexible enough to bend along the pathways of the lungs, while being inserted to the depth needed. As the airways are quite small, the medical device must have a small diameter in the distal section to be able to travel down the airways at the periphery of the lung, without damaging the lungs.

Exemplary robots work by driving or controlling wires running through conduits in the wall of the robot which are attached at the distal end, like tendons. The driving wires are connected to the distal ends of each bending section, and forces acting on the proximal end of these wires create a bending moment in that section. There can be multiple bending sections in one continuum robot, although most of the medical devices referenced only have one, since multiple bending sections increase overall size of the robot. In addition, multiple control wires increase the crosstalk effects from one bending section to another. As most manual catheters currently have one pre-bent section that the physician manually rotates to steer, added degrees of freedom are a large step from the industry standard.

The name continuum robot implies that there are no discrete rotational joints present in the device. Instead, bending is distributed over a bending section to make circular arcs, rather than sharp corners. Bending sharply would not be useful, since the point where bending occurs could damage the robot itself, could damage a tool such as an endoscopic camera upon insertion in the tool channel, or could prevent a tool from being able to be inserted through the tool channel. Even when bending in smooth circular arcs, there is a limit to how large of curvature is achievable for a particular design. For these tube-like devices there is the threat of cross-sectional collapse, or kinking, which renders the tool channel inoperable by way of constriction. Accordingly, there is a need for a robot structure which allow for a greater degree of bending, while preventing kinking.

### SUMMARY

To address such exemplary needs in the industry, the presently disclosed apparatus teaches an apparatus and method that includes a bendable body having a hollow cavity extending the length of the bendable body and a wall formed about the hollow cavity, as well as at least one control wire slideably situated in the wall and attached to the wall at a distal end of the bendable body, and at least one support wire slideably situated in the wall.

The apparatus may include an anchor at the distal end of the bendable body for attaching the control wire to the wall. In other embodiments, and the wall may include at least two lumens or hollows for slideably accommodating the at least one control wire and at least one support wire. The at least two lumens may each extend the length of the wall. The at least one support wire may be attached to the wall at the distal end of the bendable body. A second support wire may be slideably situated in the wall. The at least one support wire may be attached to a spring at the proximal end of the bendable body, with the spring being configured to alter a bending stiffness of the support wire. The at least one support wire may be attached to a driving unit at the proximal end of the bendable body, with the driving unit being configured to alter a bending stiffness of the support wire. The at least one support wire may include an outer wire and an inner wire, with the inner wire slideably nested within the outer wire. The support wire and the control wire may be formed of or include a radio opaque material. The at least one support wire may be configurable in girth, length, stiffness and position within the wall to alter a bending stiffness of the bendable body.

As aspect of the present disclosure provides an apparatus that includes a bendable body including a hollow extending a length thereof, the hollow having a wall formed about at least a part thereof and at least one support wire extending in the hollow. A distal end of the at least one support wire is anchored to the wall, with proximal portions of the at least one support wire being slideably situated in the hollow. At the proximal end of the bendable body, a proximal end of the at least one support wire is configured to attach to at least one of a spring and a force controller.

Another aspect of the present disclosure provides a continuum robot that includes a bendable body including a plurality of hollows extending a length thereof, each hollow having a wall formed about at least a part thereof; at least one control wire extending in a hollow of the plurality of hollows; and at least one support wire extending in another hollow of the plurality of hollows. A distal end of the at least one control wire is anchored to a wall of the hollow, with proximal portions of the at least one control wire slideably situated in the hollow. At a proximal end of the bendable body, a proximal end of the at least one control wire is configured to attach to a position controller. A distal end of the at least one support wire is anchored to a wall of the another hollow, with proximal portions of the at least one support wire being slideably situated in a respective hollow. At the proximal end of the bendable body, a proximal end of the at least one support wire is configured to attach to at least one of a spring and a force controller.

A further aspect of the present disclosure provides a method of treatment that includes inserting a continuum robot into one of an orifice and a fissure, and advancing the continuum robot to a target. The continuum robot includes a bendable body including a plurality of hollows extending a length thereof, each hollow having a wall formed about at least a part thereof; at least one control wire extending in a hollow of the plurality of hollows; and at least one support wire extending in another hollow of the plurality of hollows. A distal end of the at least one control wire is anchored to the wall, with proximal portions of the at least one control wire slideably situated in the hollow. A distal end of the at least one support wire is anchored to a wall of the another hollow, with proximal portions of the at least one support wire being slideably situated in the another hollow. At the proximal end of the continuum robot, a proximal end of the at least one support wire is configured to attach to at least one of a spring and a force controller. The advancing includes bending the continuum robot. During the bending, the at least one of the spring and the force controller is configured to provide a restoring force on the at least one support wire to prevent kinking.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present disclosure will become apparent from the following detailed description when taken in conjunction with the accompanying figures showing illustrative embodiments.

FIGS. 1A and 1B provide front cross-sectional and side perspective views, respectively, of a bendable medical device according to the existing art.

FIGS. 2A and 2B provide front cross-sectional and side perspective views, respectively, of a bendable medical device according to the existing art.

FIGS. 3A and 3B provide front cross-sectional and side perspective views, respectively, of a bendable medical device according to the existing art.

FIG. 4 depicts a perspective view of an exemplary bendable medical device, according to one or more embodiments of the subject apparatus, method or system.

FIG. 5 is a cut-away view of an exemplary bendable medical device inserted into a cavity, according to one or more embodiments of the subject apparatus, method or system.

FIGS. 6A and 6B are front cross-sectional and side perspective views, respectively, of an exemplary bendable medical device, according to one or more embodiments of the subject apparatus, method or system.

FIG. 7 is a side perspective view of an exemplary bendable medical device, according to one or more embodiments of the subject apparatus, method or system.

FIGS. 8A through 8F are front cross-sectional views of various exemplary bendable medical device, incorporating different support wire placements, according to one or more embodiments of the subject apparatus, method or system.

FIG. 9 is a side perspective cut-out view of an exemplary bendable medical device, according to one or more embodiments of the subject apparatus, method or system.

FIG. 10 is a side perspective cut-out view of an exemplary bendable medical device, according to one or more embodiments of the subject apparatus, method or system.

FIG. 11 is a side perspective cut-out view of an exemplary bendable medical device, according to one or more embodiments of the subject apparatus, method or system.

Throughout the drawings, the same reference numerals and characters, unless otherwise stated, are used to denote like features, elements, components or portions of the illustrated embodiments. In addition, reference numeral(s) including by the designation “ ’ ” (e.g. 12’ or 24’) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION

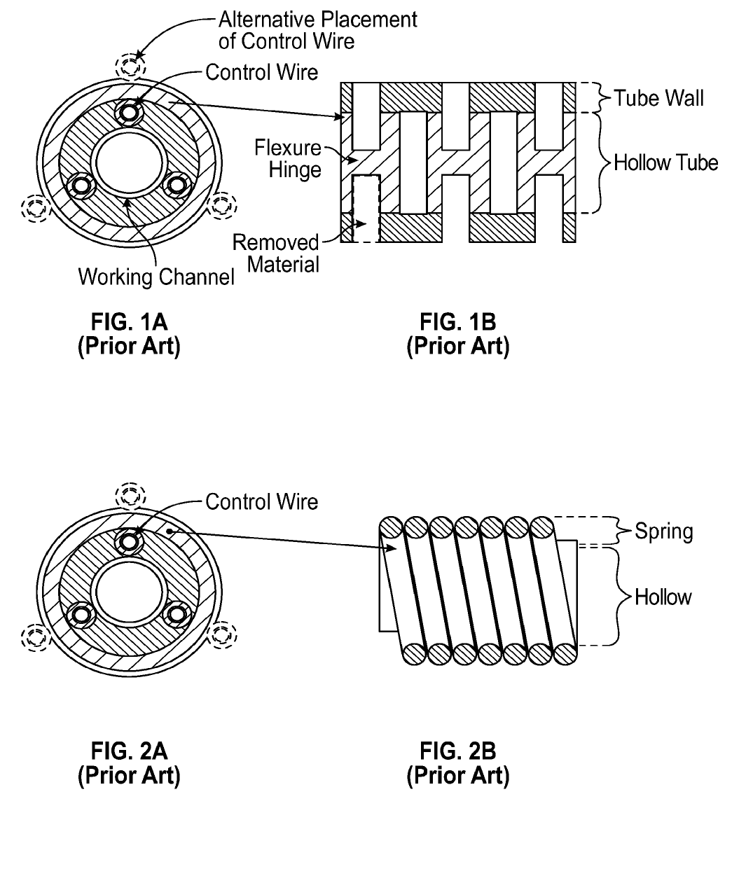

FIGS. 1 through 3 provide variants of bendable medical devices currently known in the art. FIG. 1A and 1B are illustrations of the medical device disclosed in U.S. Patent No. 7,914,466 to Davis et al., and detail a flexure-cut tube where alternating relief cuts in perpendicular axes allow the tube to bend much more easily in two degrees of freedom, while maintaining a reasonable amount of axial stiffness compared to the uncut tube. The flexure cut geometry is highly variable and can be customized for specific need and application. The flexure cut backbone is typically made from a nitinol or stainless steel hypodermic tube, which means it suffers from a challenge to miniaturization, namely that the driving wires must be routed on the inside or the outside of the tube wall. If run on the inside, the tool channel is reduced in diameter, and when run on the outside of the tube wall the overall diameter of the device is increased, which further lends support for the currently contemplated need in the field of art.

FIGS. 2A and 2B also depict a typical bendable medical device known in the art, wherein a wound, spring-like structure provides some axial stiffness due to the solid length of the coil under compression. The coil may be spring-like, where the wound wire has circular cross section, or it may be more ribbon shaped with oblong or even flat cross section. This structure again suffers from the fact that the driving wires must run on either the inside or the outside of the tube cross section, and thereby increase the outer diameter of the catheter or reduce the tool channel diameter.

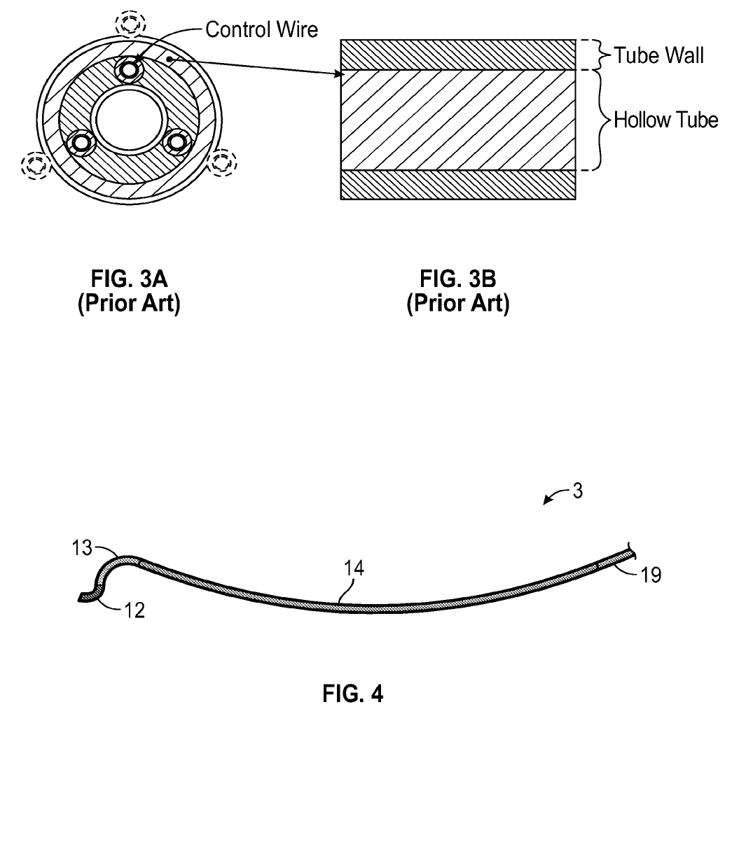

FIGS. 3A and 3B are yet another support structure used in continuum robotics, wherein a wound or braided structure is used to provide rigidity and prevent kinking. The advantage of the wound or braided structure is that the overall tube or sleeve structure is capable of high bending strain, because the individual strands of material that make up the tube are routed around the tube in a direction non-parallel to the axis of the tube, as well as the ability of the individual strands to translate relative to each other. Although this structure does not possess as much axial rigidity as the previous examples, it also suffers from the addition of cross sectional space as the driving wires must run on either the inside or the outside of the tube cross section.

The subject innovation is hereby detailed in FIGS. 4 through 11, wherein various embodiments are contemplated and disclosed. Briefly stated, the issues resolved by the subject innovation involve adding support to the structure to dissuade kinking, without adding to the cross sectional area of the bendable body, as well as without significantly adding to the bending stiffness of the bendable body. This in return allows for a high bend curvature without kinking and/or cross sectional collapse of the tub-like bendable body.

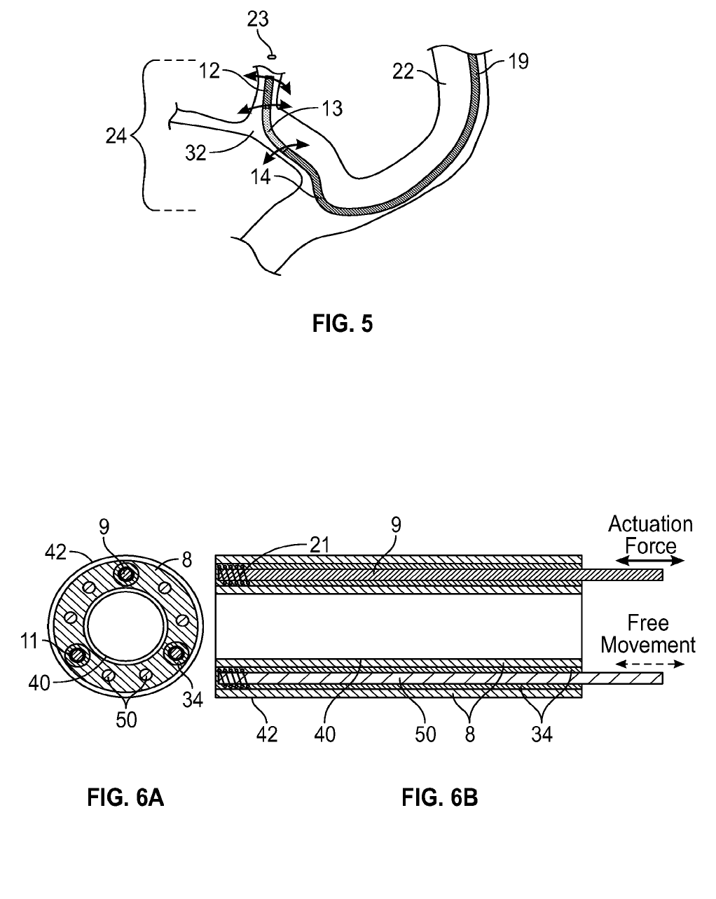

FIG. 4 is a schematic drawing to explain the bendable segments of the bendable medical device 3. The bendable medical device 3 includes a proximal part 19 and three bendable segments, which are the first, second, and third bendable segments 12, 13, 14, respectively. The bendable segments 12, 13, 14, can independently bend and can form a shape with three independent curvatures, as seen in FIGS. 4 and 5.

FIG. 5 provides a cut-away view of an exemplary bendable medical device 3 inserted into a cavity, specifically, the peri-bronchial area of a patient’s lungs, which is a lateral area surrounding the airways. This area is a known challenge to target as identified in literature, and the prior art, due to the limited distal dexterity of the conventional catheter. To reach the lesion through airways 22 in the navigation stage, the first and the second bendable segments 12, 13, respectively, navigate the bendable medical device 3 through the bifurcation point 32. The first bendable segment 12 can adjust the shape/orientation to the daughter branch while the second bendable segment 13 can adjust the shape/orientation to the parent branch in the bifurcation point 32, as the bendable medical device 3 advances through the bifurcation point 32. Once the first and the second bendable segments 12 and 13 pass the bifurcation point 32, those segments may act as guides for the rest of the bendable medical device 3, so that the insertion force from the proximal end of the bendable medical device 3 can be effectively transformed into the insertion force for a distal part of the bendable medical device 3 without serious prolapsing of the distal section. Once the distal end 24 of the bendable medical device 3 reaches the vicinity of the lesion 23, the bendable medical device 3 would direct the distal end 24 to the lesion 23, which locates the lateral area around the airway, by bending the first and the second bendable segments 12 and 13, respectively. The airway does not directly connect with the lesion 23, which is a difficult for a conventional catheter to traverse.

With the first, the second and the third bendable segments 12, 13 and 14, respectively, the bendable medical device 3 can orient the distal end 24 without moving the proximal part 19 that goes through all bifurcations to this lesion 23. By using the three-dimensional bending capability of the first and the second bendable segments 12 and 13, the bendable medical device 3 can perform unique maneuvers to enhance capability of the peri-bronchial targeting. Therefore, the bendable medical device 3 can provide improved access to the intended lesion 23 through tortuous pathways. Also, the bendable medical device 3 can have different flexibility along the axial direction without increasing the size or number of the jointing points.

In the subject embodiment depicted in FIGS. 6A and 6B, the bendable medical device 3 has an outer diameter 42 which is miniaturized to fit within the peripheral airways of the lung (approximately 3mm), while maintaining a proper sized tool channel 18 (approximately 2mm inner diameter) established by the inner diameter 40. This leaves an annular wall 8 with a thickness of approximately 0.5mm to house the control wires 9, 10, 11, and establish rigidity to prevent kinking. Each of the bendable segments 12-14 have at least two control wires 9-11, respectively, which leaves little room to spare. The constraint on the wall 8 thickness of the bendable body 7 and the existence of multiple control wires 9, 10, 11, already running through the wall 8 severely limits the cross sectional space.

In order to address the kinking issues, support wires 50 are provided in the wall 8 of the bendable body 7, and may be anchored to the distal end 24 of a bending segment 12-14. The support wires 50 may run through lumens 34 configured in the wall 8, which may originate at the proximal part 19 of the bendable medical device 3. In certain embodiment, the support wires 50 in the distal bending sections run through the proximal part 19 to provide similar benefit. In one embodiment, all the support wires 50 may extend from the distal end 24 of the bendable medical device 3 to the proximal part 19 of the bendable medical device 3, thus allowing all segments 12-14 of the bendable body 7 to gain the kink prevention benefits.

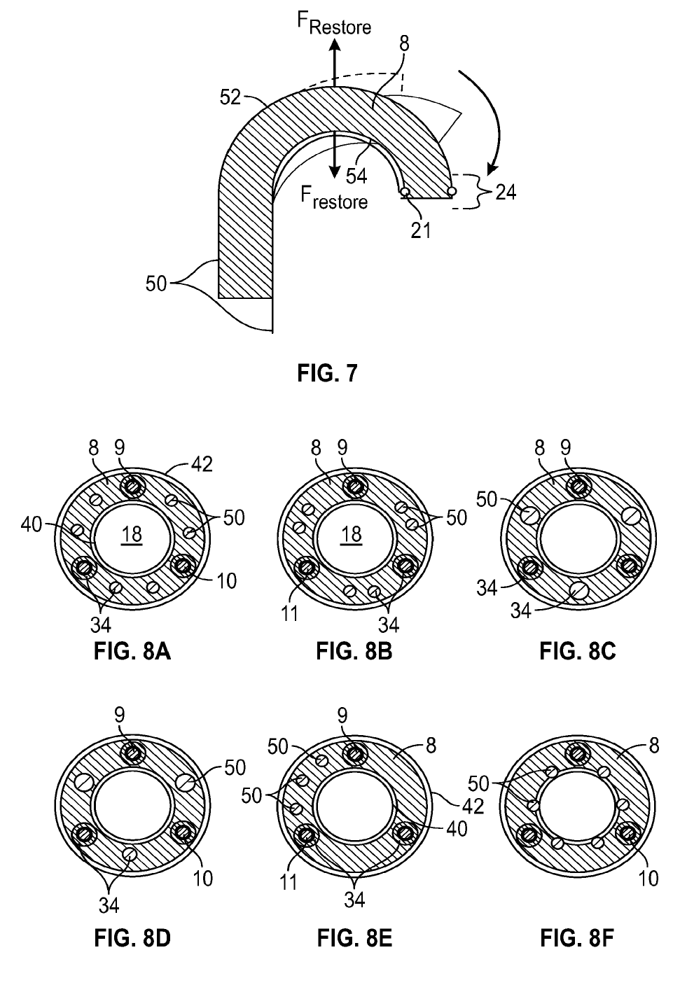

In application, as the bendable medical device 3 bends, the support wires 50 prevent the cross section of the tube from collapsing or kinking, by providing a restoring force against the tendency for the wall 8 to flatten. As depicted in FIG. 7, a bendable body 7 is bent with two support wires 50 attached at the distal end 24 of the wall 8, while allowing the support wires 50 to freely slide through their respective lumens 34. When the bendable body 7 is bent by external forces, the length of the two opposing top surface 52 and bottom surface 54 of the bendable body 7 change, due to their offset from the neutral bending axis. Because the support wires 50 are only fixed at one end of the wires 50, in this example the distal end 24, the support wires 50 are free to slide within the respective lumens 34. If the support wires 50 were attached at both the proximal part 19 and distal end 24, the support wires would have to change length to match the change in length for the top surface 52 and bottom surface 54 of the bendable body 7. Additionally, when the support wires 50 are bent, but not axially strained as is this case, the bent support wires 50 may provide a restorative force.

FIGS. 8A–8F depict cross-sectional images that show various configurations for placement of the support wires 50, relative to the control wires 9-11, and within the wall 8 of the bendable body 7. In addition, the number, size and placement of the support wires 50 can be varied to provide specific mechanical properties, such as bending stiffness in a particular direction. The ability to customize the bending stiffness with respect to the bending direction is yet another very advantageous effect when designing a complicated structure that has asymmetry. The figures show several examples of how the number, size and position of the support wires 50 can be varied to produce the desired mechanical effect. The control wires 9-11, wall 8, lumens 34 and tool channel 18 are for ease of understanding. As can be seen, noncircular, nonconcentric structures with any number of control wires 9-11 located at any position in the cross section will also benefit from the design advantage that the support wires 50 provide. FIG. 8A provides a symmetric layout with equally spaced, equally sized support wires 50. FIG. 8B depicts a symmetric layout of densely spaced, equally sized support wires 50. FIG. 8C shows symmetrically placed large support wires 50. FIG. 8D provides mixed sized support wires 50, with FIG. 8E showing asymmetrically placed support wires 50. Finally, FIG. 8F depicts support wires 50 placed offset from the control wire 9-11 radius.

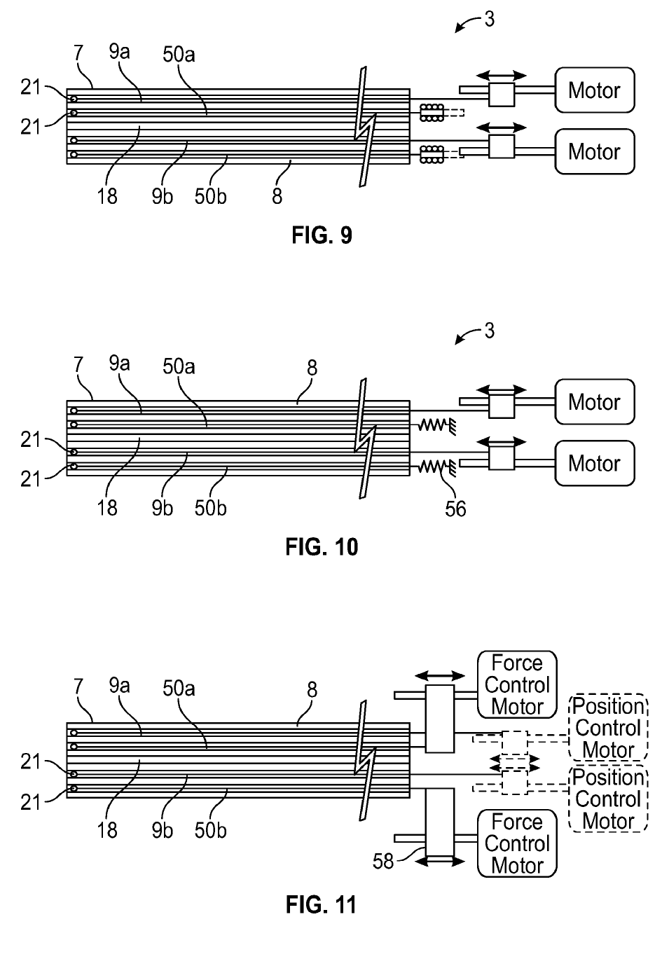

FIG. 9 illustrates a side perspective cut-out view of an exemplary bendable medical device 3, according to one or more embodiments of the subject apparatus, method or system. The exemplary bendable body 7 has four lumens 34 and a central tool channel 18. Each of the four lumens 34 is occupied by two control wires 9a and 9b, as well as two support wires 50a and 50b. The support wires 50 are free to translate inside the respective lumens 34, while being anchored by the anchors 21 to the body 7 at the distal ends 24 of the bendable body 7. This represents one exemplary embodiment of the subject innovation, where the support wires 50 simply bend along with the wall 8 structure in a passive manner. This configuration provides minimal added flexural rigidity to the overal bendable medical device 3 structure while adding to the longitudinal stiffness and preventing kinking or binding.

FIG. 10 illustrates a side perspective cut-out view of an exemplary bendable medical device 3, according to one or more embodiments of the subject apparatus, method or system. Similar to FIG. 9, except the proximal ends of the support wires 50 are attached to the proximal end of the bendable medical device 3 by springs 56. The benefit of this embodiment is that the support wires 50 can be elected for preloading, by the springs 56, or be set to a neutral position, allowing for adjustablility based on the desired stiffness required by the end user. The bending medical device 3 becomes marginally stiffer in bending, which in turn allows the bending medical device 3 to more easily straighten itself when driving wire forces are neutralized.

FIG. 11 provides a side perspective cut-out view of an exemplary bendable medical device 3, according to one or more embodiments of the subject apparatus, method or system. In this embodiment, the proximal ends of support wires 50 are attached at the proximal part 19 of the bendable medical device 3, and are actuated at the proximal part 19 by independent force control modules 58, such that the total force acting longitudinally on the support wires 50 is negligible. In this way, the bendable medical device 3 becomes more resilient to kinking while providing minimal extra bending stiffness. Once again, to allow for fine adjustment of the bending stiffness while eliminating kinking and binding.

The present disclosure allows the structure to be incorporated into miniaturized medical devices and continuum robots without the need for an additional cross section support and added girth. The support structure is integrated within the existing wall and in cooperation with the required driving wires, wherein the support wires can be similarly formed, at the same time, and to cooperate with existing control wires.

In addition, the number, placement, rigidity and size of support wires can be selected to customize the additional bending stiffness of the bendable medical device 3 to suit varying requirements. This is advantageous when the bendable medical device structure already has asymmetric features. For bending sections that have two degrees of freedom (e.g., bend about the x and y direction vectors) the ability to bend the bendable medical device uniformly in any direction is a high priority. The support wire structure allows the bendable medical device to have greater design freedom. Likewise, the placement of support wires can be customized to control the overall bending stiffness and axial stiffness (for insert ability) of each bending section.

1. An apparatus comprising:
a bendable body including a hollow extending a length thereof, the hollow having a wall formed about at least a part thereof; and
at least one support wire extending in the hollow, wherein:
a distal end of the at least one support wire is anchored to the wall, with proximal portions of the at least one support wire being slideably situated in the hollow, and
at the proximal end of the bendable body, a proximal end of the at least one support wire is configured to attach to at least one of a spring and a force controller.

2. The apparatus of claim 1, wherein the at least one support wire is configured to provide a restoring force to prevent kinking of the bendable body.

3. The apparatus of claim 1, wherein the at least one of the spring and the force controller is configured to alter a stiffness of the bendable body.

4. The apparatus of claim 1, wherein the at least one of the spring and the force controller is configured to preload the at least one support wire to set one of a neutral position and a stiffness of the bendable body.

5. The apparatus of claim 1, wherein the at least one support wire is configurable in at least one of girth, length, stiffness and position within the hollow to alter a stiffness of the bendable body.

6. The apparatus of claim 1, wherein the at least one support wire comprises an outer wire and an inner wire, with the inner wire slideably nested within the outer wire.

7. The apparatus of claim 1, further comprising:
a tool channel extending at least a portion of the length of the bendable body; and
another support wire, wherein the at least one support wire and the another support wire are symmetrically and/or equidistantly arranged around the tool channel.

8. The apparatus of claim 7, wherein the tool channel is configured to accommodate a tool configured to perform one of imaging, biopsy, and a medical procedure.

9. The apparatus of claim 8, wherein:
the tool is configured to be manipulated to extend through the tool channel from at or adjacent to the proximal end of the bendable body to a distal end of the bendable body, and
the bendable body is configured to provide flexible access within a patient for the tool to perform a medical procedural.

10. The apparatus of claim 1, further comprising:
another hollow extending the length of the bendable body, the another hollow having a wall formed about at least a part thereof; and
at least one control wire extending in the another hollow, wherein:
a distal end of the at least one control wire is anchored to the wall of the another hollow, with a proximal portion slideably situated in the another hollow, and
at a proximal end of the bendable body, a proximal end of the at least one control wire is configured to attach to at least one position controller.

11. A continuum robot comprising:
a bendable body including a plurality of hollows extending a length thereof, each hollow having a wall formed about at least a part thereof;
at least one control wire extending in a hollow of the plurality of hollows; and
at least one support wire extending in another hollow of the plurality of hollows, wherein:
a distal end of the at least one control wire is anchored to a wall of the hollow, with proximal portions of the at least one control wire slideably situated in the hollow,
at a proximal end of the bendable body, a proximal end of the at least one control wire is configured to attach to a position controller,
a distal end of the at least one support wire is anchored to a wall of the another hollow, with proximal portions of the at least one support wire being slideably situated in a respective hollow, and
at the proximal end of the bendable body, a proximal end of the at least one support wire is configured to attach to at least one of a spring and a force controller.

12. The continuum robot of claim 11, wherein the at least one support wire is configured to provide a restoring force to prevent kinking of the bendable body.

13. The continuum robot of claim 11, wherein the at least one of the spring and the force controller is configured to alter a stiffness of the bendable body.

14. The continuum robot of claim 11, wherein the at least one of the spring and the force controller is configured to preload the at least one support wire to set one of a neutral position and a stiffness of the bendable body.

15. The continuum robot of claim 11, wherein the at least one support wire is configurable in at least one of girth, length, stiffness and position within the respective hollow to alter a stiffness of the bendable body.

16. The continuum robot of claim 11, wherein the at least one support wire comprises an outer wire and an inner wire, with the inner wire slideably nested within the outer wire.

17. The continuum robot of claim 11, further comprising:
a tool channel extending at least a portion of the length of the bendable body; and
another support wire, wherein the at least one support wire and the another support wire are symmetrically and/or equidistantly arranged around the tool channel.

18. The continuum robot of claim 17, wherein:
the tool channel is configured to accommodate a tool configured to perform one of imaging, biopsy, and a medical procedure, and
the tool is configured to be manipulated to extend through the tool channel from at or adjacent to the proximal end of the bendable body to a distal end of the bendable body.

19. A method of treatment comprising:
inserting a continuum robot into one of an orifice and a fissure; and
advancing the continuum robot to a target,
wherein the continuum robot includes:
a bendable body including a plurality of hollows extending a length thereof, each hollow having a wall formed about at least a part thereof;
at least one control wire extending in a hollow of the plurality of hollows; and
at least one support wire extending in another hollow of the plurality of hollows,
wherein a distal end of the at least one control wire is anchored to the wall, with proximal portions of the at least one control wire slideably situated in the hollow,
wherein a distal end of the at least one support wire is anchored to a wall of the another hollow, with proximal portions of the at least one support wire being slideably situated in the another hollow,
wherein at the proximal end of the continuum robot, a proximal end of the at least one support wire is configured to attach to at least one of a spring and a force controller,
wherein the advancing includes bending the continuum robot, and
wherein, during the bending, the at least one of the spring and the force controller is configured to provide a restoring force on the at least one support wire to prevent kinking.

20. The method of claim 19, wherein:
the one of the orifice and the fissure is at least one of an airway, a trachea and an anatomical fissure, and the target is located within a patient, and
the continuum robot includes a tool channel configured to accommodate a tool configured to perform one of imaging, biopsy, and a medical procedure.
